(19) United States
(12) Patent Application Publication
(10) Pub. No.: US 2024/0374647 A1
(43) Pub. Date: Nov. 14, 2024

(54) APPLICATION OF SPHERE SUBSTANCE IN PREPARATION OF INTRAVENOUS INJECTION

(71) Applicants: UNIVERSITY OF MACAU, Macau (CN); YUNNAN KEY LABORATORY OF PRIMATE BIOMEDICAL RESEARCH, Kunming (CN)

(72) Inventors: Ren-He XU, Macau (CN); Wei SI, Kunming (CN); Yaping YAN, Kunming (CN); Changkun YANG, Macau (CN); Enqin LI, Macau (CN)

(21) Appl. No.: 18/576,751

(22) PCT Filed: Apr. 21, 2022
(86) PCT No.: PCT/CN2022/088276

§ 371 Date: Jan. 5, 2024

(30) Foreign Application Priority Data
Jul. 7, 2021 (CN) 202110765691.4
Mar. 17, 2022 (CN) 202210265785.X

(51) Int. Cl. A61K 35/28

(52) U.S. Cl. CPC A61K 35/28

(57) ABSTRACT

Provided is an application of a sphere substance in the preparation of an intravenous injection, relating to the technical field of biomedicine. Intravenous injection of the sphere substance formed by MSC spheres or biomaterials is used as a new in-vivo delivery solution. Compared with the intravenous injection of discrete MSCs, the intravenous injection of the MSC spheres reduces the retention of the MSCs in lungs, and prolongs the-retention time and activity of the MSCs in blood and peripheral tissues, such that therapeutic effects for autoimmune diseases, inflammatory diseases, and tissues and organ injuries can be improved. In addition, the intravenous injection of biomaterial spheres is safe, can carry drugs, biologically active molecules or detection molecules, is applied to diagnosis and treatment of an animal disease model or a patient, is more effective and more durable than monomer molecules, and can also carry a variety of molecules to generate a synergistic effect.

### CROSS-REFERENCES TO RELATED APPLICATIONS

This application claims the priority of the Chinese patent applications entitled “The Application of Mesenchymal Stromal Cell Spheroid in the Preparation of Intravenous Injections” with application Ser. No. 202110765691.4, submitted to the China National Intellectual Property Administration on Jul. 7, 2021 and “The Application of Spherical Materials in the Preparation of Intravenous Injections” with application Ser. No. 202210265785.X, submitted to the China National Intellectual Property Administration on Mar. 17, 2022. The entire contents of these applications are incorporated herein by reference.

### TECHNICAL FIELD

The present invention relates to the field of biopharmaceutical technology, and more specifically, to the application of spheroidal materials in the preparation of intravenous injections.

### BACKGROUND TECHNIQUE

The stem cell characteristics of mesenchymal stromal cells (MSCs) are mainly demonstrated via in vitro culture. Either in vitro or in vivo experiments, their properties of anti-inflammatory and pro-inflammatory are majorly exerted by secreting factors that regulate immunity and induce regeneration, with a minority relying on differentiation into specific cell types. Therefore, MSCs have been widely applied in animal models and clinical trials. In comparison to intramuscular, intra-articular, intracardiac, intramedullary, intrathecal, and local injection into skin wounds, intravenous injection (IV) of MSCs allows for systemic delivery via the bloodstream, providing a simple and reproducible approach that satisfies the requirements for treating various diseases. Due to the first-pass effect, IV-injected MSCs predominantly accumulate in the lungs and rapidly disappear post-injection. Nevertheless, the retained MSCs in the lungs can still exert therapeutic effects by primarily secreting cytokines, small molecules, and extracellular vesicles, thereby acting upon target cells within the lung or immune cells circulating through the pulmonary vasculature. The enrichment of MSCs in the lungs is believed to be advantageous for addressing diverse pulmonary inflammations caused by pathogens such as Covid-19.

It is widely recognized that dissociated cells suspended in liquid are the common form used for intravenous injections. However, when MSCs are administered via intravenous injection, they are quickly cleared by the body, which may be associated with their limited and unstable therapeutic efficacy observed in clinical trials. Consequently, the efficacy of MSCs as cellular therapies in clinical applications is unsure.

In view of these circumstances, the present invention is proposed.

### CONTENT OF INVENTION

The purpose of this invention is to provide the application of spherical materials in the preparation of intravenous injections.

This invention is fulfilled as follows:
Firstly, an embodiment of this invention provides the application of spherical materials in the preparation of intravenous injections, where the spherical materials are selected from at least spheroids formed by mesenchymal stromal cells (MSCs) or biomaterials with similar or identical size to a MSC spheroid.

The present invention has the following beneficial effects:
This invention proposes for the first time that intravenous injection of MSC spheroids or other biomaterials formed into similarly sized spherical matter as a new drug delivery strategy for large animals. Compared with the intravenous injection of dissociated MSCs, the intravenous injection of MSC spheroids reduces the retention of MSCs in the lungs and prolongs the survival time and viability of MSCs in the blood and peripheral tissues, thereby enhancing their therapeutic effects. Especially surprising is the finding that hosts (monkeys) receiving intravenous injection of MSC spheroids showed no adverse reactions or lethal events. Therefore, this discovery provides a new method for research and clinical application of spheroids formed by MSCs or biomaterials.

### FIGURE LEGENDS

To better illustrate the technical scheme of the present invention, a brief introduction to the figure attachments necessary for the implementation examples will be provided below. The following attachments only show some embodiments of this invention and should not be considered as limitation of scope. For those skilled in the technology, other relevant diagrams can be obtained from these attachments without the input of creative labor.

FIG. 1: Identification of Mesenchymal stromal cells (MSCs) in implementation example 1. The flow cytometry results show that both cell lines express typical MSC surface markers and do not express hematopoietic cell markers. Oil Red O, alcian Blue, and alizarin red staining verifies that both cell lines have trilineage differentiation potential. Scale bar, 100 μm.

FIG. 2: The proliferation and senescence of mesenchymal stromal cells (MSCs) in implementation example 1. Ki67⁺ cells are stained with red fluorescence, GFP⁺ cells in green, and cell nuclei are counterstained with DAPI. Senescent cells are identified by β-gal staining in light green. Scale bar, 100 μm.

FIG. 3: The preparation of MSC spheroids in implementation example 1. Observed under the fluorescence microscope, GFP⁺ MSC is prepared from monolayer cells into MSC spheroids using hanging drop method. Scale bar, 500 μm.

FIG. 4: Identification of MSC after spheroid formation in implementation example 1. CD73⁺ cells are represented by red fluorescence in the cytoplasm (upper row), and Ki67⁺ cells are represented by red fluorescence in the nucleus (lower row). Cell nuclei are counterstained with DAPI in blue. Scale bar, 150 μm.

FIG. 5: A scheme illustrating the injection of dissociated cells and cell spheroids in implementation example 1.

FIG. 6: A scheme of the animal experimental process in implementation example 1.

FIG. 7: The indices of red blood cells, platelets, and hemoglobin in monkey blood before and after MSC injection in implementation example 1.

FIG. 8: The indices and proportions of various types of leukocytes in monkeys' blood before and after MSC injection in implementation example 1.

FIG. 9: The results of biochemical analysis of monkey blood before and after MSC injection in implementation example 1.

FIG. 10: The CT scan of monkey lungs before and after MSC injection in implementation example 1.

FIG. 11: The immunofluorescent double staining of GFP and α-SMA in monkey lungs and heart 1 hour (1 h) and 21 days (D21) after MSC injection in implementation example 1. GFP⁺ cells are stained in red, α-SMA⁺ cells in green, and cell nuclei are counterstained with DAPI in blue. Scale bar, 100 μm.

FIG. 12: The proportion of cells with human-specific sequences detected in different tissues of monkeys 1 h after MSC injection in implementation example 1.

FIG. 13: The expression of integrin in MSC spheroid (MSCₛₚ) and dissociated MSC (MSC_{diss}) in implementation example 1. ITGA5, ITGB1 and ITGav are stained in red and cell nuclei are counterstained with DAPI in blue. Scale bar, 50 μm.

FIG. 14: The in vitro fluid shear stress experiments of MSC_{diss} and MSCₛₚ in implementation example 1. Panel A is YOPRO1/PI double staining and morphology of MSC_{diss} and MSCₛₚ before and 1 h after fluid shear stress circulation. Apoptotic cells are stained by YOPRO1 in green and necrotic cells are stained by PI in red. Panel B shows the statistics of YOPRO1/PI double staining before and after circulation. Scale bar, 100 μm.

FIG. 15: The intravenous injection of fluorescent methacrylate hydrogel microspheroids in monkeys for implementation example 1. Panel A shows the morphology of the hydrogel microspheroids. Panel B indicates the average size of the hydrogel microspheroids. Panel C illustrates the experimental design of the IV injection of hydrogel microspheroids. Panel D represents the state of the monkey before and 14 days after the hydrogel microspheroids injection. Panel E shows the indices of monocytes, lymphocytes, and neutrophils in the monkey blood before and after the hydrogel microspheroids injection; Panel F depicts the indices of red blood cells, platelets, and hemoglobin in the monkey blood before and after the injection of the hydrogel microspheroids; Panel G presents the results of biochemical analysis of monkey blood before and after the hydrogel microspheroids injection. Scale bar, 100 μm.

### DETAILED IMPLEMENTATION

To make the objectives, technical solutions, and advantages of the present invention clearer, the technical method of invention will be described clearly and completely below. If the conditions are not specified in implementation, they should be carried out according to the conventional conditions or recommended by the manufacturers. If the manufacturers of the reagents or instruments used are not indicated, they are all conventional products that can be purchased commercially.

The present invention provides the application of spheroidal materials in the preparation of intravenous injections. The spheroidal materials are selected either from the mesenchymal stromal cell spheroids or spheroidal materials formed from biological materials in the same or similar size.

The “biomaterials” in this invention can be selected from hydrogel, hyaluronic acid, chondroitin sulfate, etc.

The term “MSC spheroid” in this invention refers to clusters of multiple mesenchymal stromal cells (MSCs) that aggregate in a suitable culture medium under specific conditions. The conditions for MSC spheroid formation can be implemented using existing methods if not specified.

Compared to dissociated MSC (MSC_{diss}), MSC spheroids (MSCₛₚ) have a larger diameter. It is generally believed that the circulation of injected cell spheroids in blood vessels is poorer, which may cause vascular embolism in the recipient and lead to sudden death. Therefore, to date, there have been no reports on the application of cell spheroids for intravenous injection.

The administration of the intravenous injection is intended for animals or humans. Preferably, large animals, include but are not limited to sheep, horses, cows, rabbits, monkeys, and camels.

Through a series of creative work, this invention has been proposed and validated that intravenous injection of cell spheroids in animals is safe. Prior to this, no one has proposed relevant technological insights. This is a new administration scheme for MSCs, which reduces the lung retention of MSCs compared to traditional injections of dissociated MSCs. It prolongs their residence time and vitality in the blood and peripheral tissues, thereby improving efficacy. It provides a new approach and research method for the clinical application of MSCs.

The injection of other spherical biomaterials is also safe and can be applied for the delivery of specific functional molecules for the diagnosis and treatment of specific diseases. This provides a new research direction and approach for the effective diagnosis and treatment of diseases.

The spheroidal size range of mesenchymal cell spheroid can be determined based on the diameter of the recipient's vein (the site for intravenous injection), and it should be smaller than the diameter of the recipient's vein. Preferably, the size of the mesenchymal stromal cell spheroids is in the range of 200 to 800 μm. It suggests that this range applies to recipients with venous diameters of 1000 μm (or 1 mm) and above, such as the cynomolgus macaques (note: the average venous diameter in humans is 2-10 mm). The inventors herein have found that when MSC spheroids were injected intravenously and euthanized on the 21st day after injection, there were no deaths among the recipient animals, and no significant behavioral, physiological differences, or other adverse reactions were observed before and after intravenous injection. Within this range, while ensuring safety, the injection of MSC spheroids maintains their therapeutic advantages compared to dissociated MSCs.

Specifically, the diameter of a mesenchymal stromal cell spheroid can be any value among 100 μm [symbol] 110 μm [symbol] 120 μm [symbol] 130 μm [symbol] 140 μm [symbol] 150 μm [symbol] 160 μm [symbol] 170 μm [symbol] 180 μm [symbol] 190 μm [symbol] 200 μm [symbol] 210 μm [symbol] 220 μm [symbol] 230 μm [symbol] 240 μm [symbol] 250 μm [symbol] 260 μm [symbol] 270 μm [symbol] 280 μm [symbol] 290 μm [symbol] 300 μm [symbol] 310 μm [symbol] 320 μm [symbol] 330 μm [symbol] 340 μm [symbol] 350 μm [symbol] 360 μm [symbol] 370 μm [symbol] 380 μm [symbol] 390 μm [symbol] 400 μm [symbol] 410 μm [symbol] 420 μm [symbol] 430 μm [symbol] 440 μm and 450 μm.

Preferably, the diameter of the MSC spheroid ranges from 200 μm to 800 μm. This range provides better technical effect for individuals with venous diameters similar to those of cynomolgus macaques.

It can be understood that the spherical biomaterial that is the same or similar in size to the MSC spheroid, referring to the same size as the MSC spheroid, or the size difference is ≤20%.

MSCs can be administered intravenously (IV) through the bloodstream, allowing for systemic delivery. This method is simple and reproducible, making it suitable for various disease treatments.

Preferably, the intravenous injection is used for the treatment of autoimmune diseases and inflammatory damage to tissues and organs.

Preferably, autoimmune diseases, including multiple sclerosis.

Preferably, inflammatory damage to tissues and organs, including COVID-19 pneumonia.

The application provided by this invention is applicable to any type of MSC, whether derived from the body or cultured cells.

Specifically, the MSC spheroids include T-MSC spheroid derived from pluripotent stem cells, such as embryonic stem cells, which can be obtained through known experimental methods. UC-MSC spheroid derived from the fetal tissue umbilical cord (UC). Adipose MSC spheroid derived from the adult tissue fat. BM-MSC spheroid derived from adult tissue bone marrow (BM).

Please note that the process of deriving T-MSCs from embryonic stem cells is not further elaborated here.

Optionally, the intravenous injection formulation may include additives.

Optionally, the additives can be selected from osmotic regulators, pH regulators, diluents, and cell protectants.

Optionally, osmotic regulators can include at least one of sodium chloride, glucose, and glycerol.

Optionally, pH regulators can include any one of hydrochloric acid, citric acid, sodium hydroxide, potassium hydroxide, sodium bicarbonate, disodium hydrogen phosphate, and monosodium dihydrogen phosphate.

Optionally, diluents can include at least one of normal saline and glucose solution.

Further detailed description of the features and performance of the present invention will be provided in the following embodiments.

### Implementation Example 1

### 1. Characterization of MSC

In this protocol, T-MSCs were derived from the Envy human embryonic stem cell (hESC) line. Briefly, hESCs were seeded at a density of 2×10⁴−6×10⁴ cells per well in a 6-well plate and cultured in mTeSR1 medium for one day. The next day, after removing spent medicells were washed with phosphate-buffered saline (PBS), and then nourishing induction medium (mTeSR-SF+1 μM A83-01 and 10 ng/mL BMP4) was added. The medium was refreshed every 2 days until day 5. On day 5, cells were washed with PBS and digested for passaging at a 1:1 ratio. The cells were then cultured in MSC medium, and the medium was refreshed every 2-3 days. Passaging was performed every 7-10 days until MSC-like cells appeared, followed by expansion and characterization. This protocol refers to Wang, X., et al., Immune modulatory mesenchymal stromal cells derived from human embryonic stem cells through a trophoblast-like stage. Stem Cells, 2016. 34 (2): p. 380-91. The T-MSCs generated in this protocol express enhanced green fluorescent protein (EGFP).

Meanwhile, in this implementation, UC-MSCs were isolated from donated fetal umbilical cords and transduced with a lentiviral vector expressing GFP. GFP UC-MSC was then established through flow cytometry sorting.

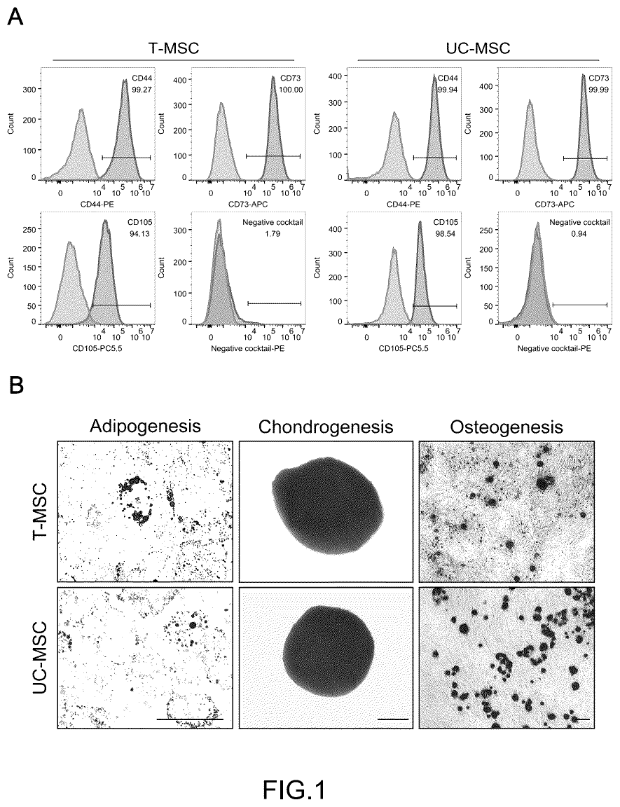

Both T-MSC and UC-MSC expressed typical MSC markers CD73, CD44, and CD105, but did not express hematopoietic cell markers CD34 and CD45 and exhibited trilineage differentiation potential (FIG. 1). Both types of MSC were passaged every 5-6 days.

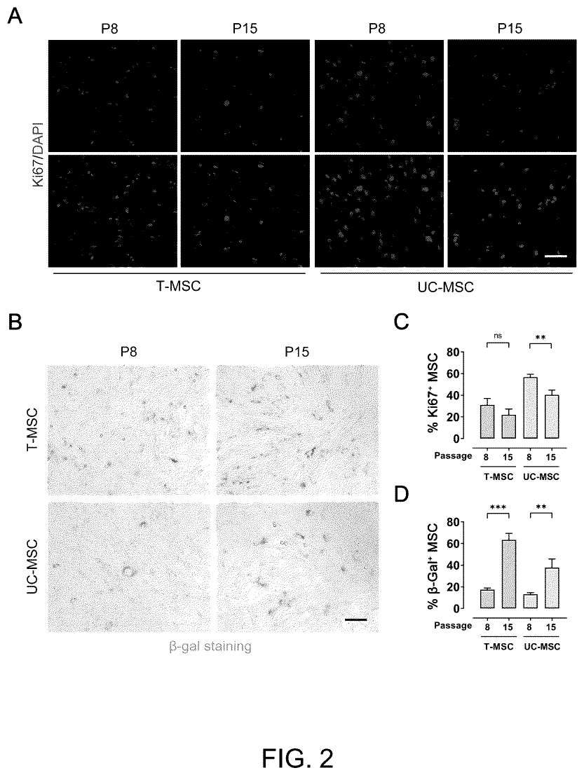

From the Ki67⁺ cell rate at the 8th passage (P8), it can be observed that UC-MSCs were more proliferative compared to T-MSC. UC-MSC accounted for approximately 50%, while T-MSCs accounted for approximately 30%. The number of proliferating cells in both MSC types decreased when cultured up to the 15th passage (P15). Additionally, based on the senescence-related β-gal⁺ cell rate, starting from P8, UC-MSC had fewer senescent cells than T-MSC, with UC-MSC accounting for approximately 15% and T-MSC accounting for approximately 20%. The β-gal⁺ cells in both MSC types significantly increased at P15 (FIG. 2).

### 2. Preparation of MSC Spheroid

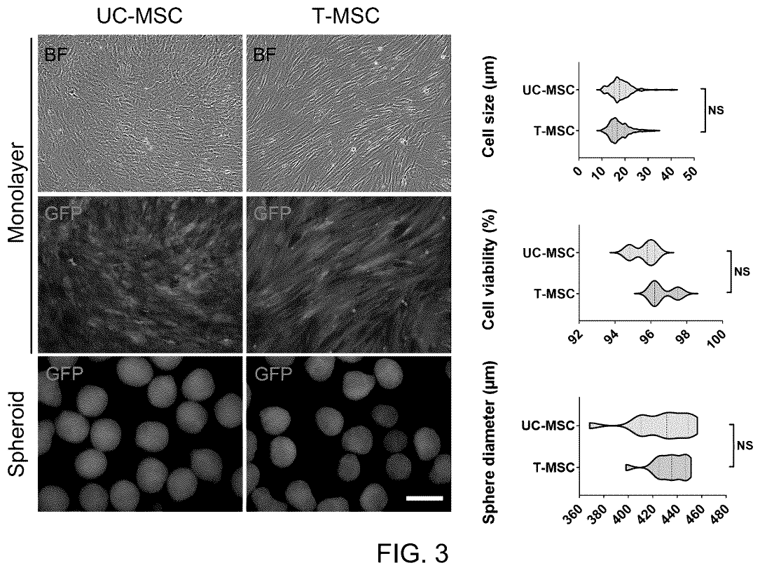

Cell spheroids were prepared using MSCs with a passage number before P10. Both types of MSCs were cultured until confluency reached 90%. After digestion, when the cell viability reached 95%, cell spheroids were prepared using the hanging drop method. In brief, individual cells obtained from digestion were resuspended in MSC culture medium and dispensed at a density of 2×10⁴/20 μL/drop on the inner side of a 15 cm culture dish lid. The lid was then placed back onto the culture dish and incubated at 37° C. with 5% CO2 for 72 hours, allowing the cells to self-assemble into cell spheroids. (Refer to Jiang, B., et al., Spheroidal formation preserves human stem cells for prolonged time under ambient conditions for facile storage and transportation. Biomaterials, 2017. 133: p. 275-286.) The average cell size of UC-MSCs was 18 μm, while that of T-MSCs was 17 μm. After spheroid formation, the diameter of the spheroids was approximately 450 μm, and there was no significant difference in size between the two types of MSC spheroids (FIG. 3).

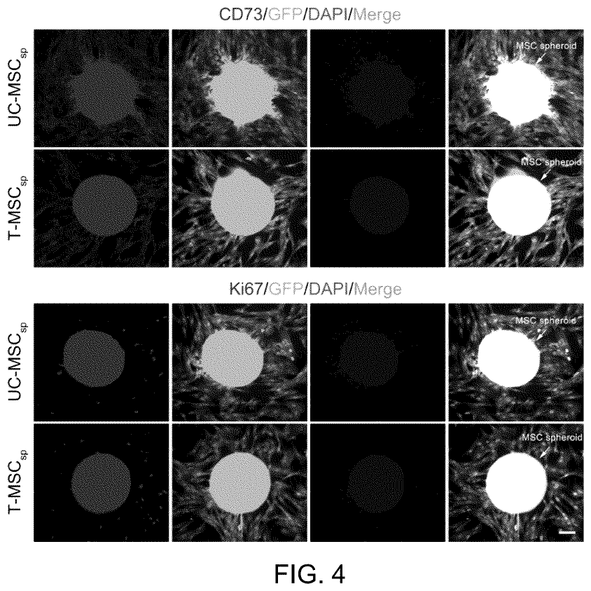

After being stored at room temperature in a low-oxygen environment for 7 days, the cell spheroids were reseeded in culture dishes. Both UC-MSC spheroids (UC-MSCₛₚ) and T-MSC spheroids (T-MSCₛₚ) were able to reattach, migrate, and expand, forming a monolayer of cells with MSC phenotype CD73. Through Ki67 immunofluorescence staining, it was observed that the reattached MSCs retained their proliferative capacity. These results indicate that after 7 days of storage at room temperature in a low-oxygen environment, MSC spheroids still maintained their MSC characteristics and proliferative ability, and the formation of cell spheroids did not impair the phenotype and function of UC-MSCₛₚ and T-MSCₛₚ (FIG. 4).

### 3. Animal Sectionalization and Cell Injection

Nine healthy cynomolgus macaques, aged 8-13 years (with an average age of 10.9 years) and weighing 2.90-4.60 kg (with an average weight of 3.68 kg), were randomly divided into three groups: Group A, Group B, and Group C. Each group received intravenous injections of different types of MSC_{diss} or MSCₛₚ. The number of cells injected was calculated based on the weight of monkeys, with a dosage of 5×10⁶ cells/kg for MSC_{diss} or MSCₛₚ (as shown in Table 1).

TABLE 1
Animal information and sectionalization

| | | | | Number of | |
|---|---|---|---|---|---|
| | | | | dissociated | |
| | | Age | Weithg | MSC/MSC | |
| | No. | (year) | (kg) | spheroids | Endpoint |
| UC-MSCₛₚ | A1 | 8 | 2.80 | 1.40 × 10\hat{ }7/700 | 1 h |
| | A2 | 13 | 3.95 | 1.98 × 10\hat{ }7/988 | D 21 |
| | A3 | 12 | 3.25 | 1.93 × 10\hat{ }7/963 | D 21 |
| T-MSCₛₚ | B1 | 11 | 3.00 | 1.50 × 10\hat{ }7/750 | 1 h |
| | B2 | 12 | 3.85 | 1.93 × 10\hat{ }7/963 | D 21 |
| | B3 | 12 | 3.35 | 1.68 × 10\hat{ }7/838 | D 21 |
| T-MSC_{diss} | C1 | 8 | 2.90 | 1.45 × 10\hat{ }7 | 1 h |
| | C2 | 13 | 4.60 | 2.30 × 10\hat{ }7 | D 21 |
| | C3 | 9 | 5.45 | 2.73 × 10\hat{ }7 | D 21 |

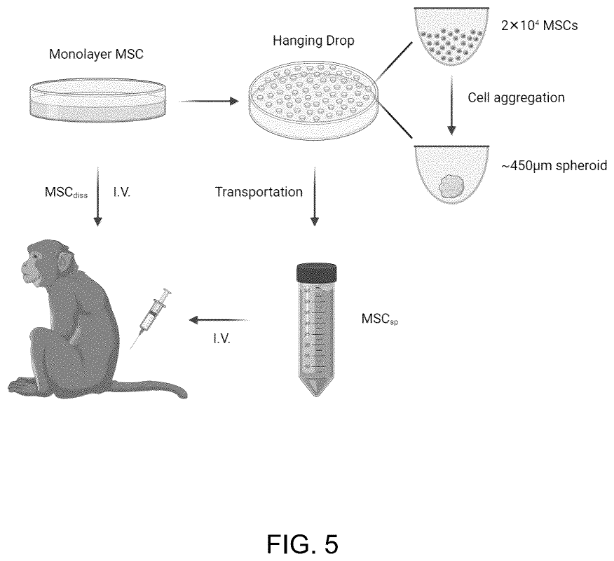

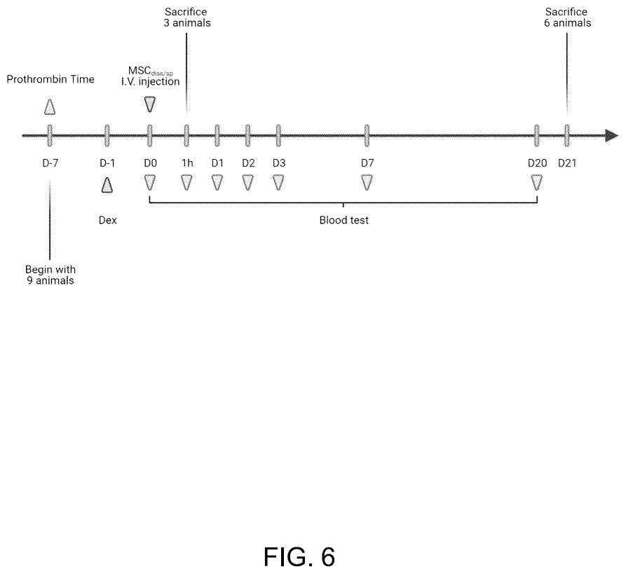

In each group, one animal was euthanized 1 hour after injection, and the remaining two animals were euthanized 21 days after injection. All animals underwent blood tests prior to the injections. To prevent allergic reactions, a muscle injection of 10 mg/kg dexamethasone was administered one day before the injection (D-1). Except for the three monkeys euthanized at 1-hour post-injection, all other animals were subjected to blood tests on the day of cell injection (D0), 1-hour post-injection, D1, D2, D3, D7, D14, and D20, and then euthanized on D21 (as shown in FIG. 5 and FIG. 6).

### 4. Live Animal Test

Before cell injection, all monkeys were in a normal physiological state without any apparent abnormalities. Seven days prior to the injection (D-7), the blood tests of nine monkeys were normal, including prothrombin time (PT) and PT/international normalized ratio (INR). The INR value is calculated based on PT to monitor the effectiveness of anticoagulant drug warfarin in preventing blood clotting. Activated partial thromboplastin time (APTT) and fibrinogen (FIB) concentration were also measured (Table 2).

The results indicate that there were no issues with coagulation in the animals prior to the injection.

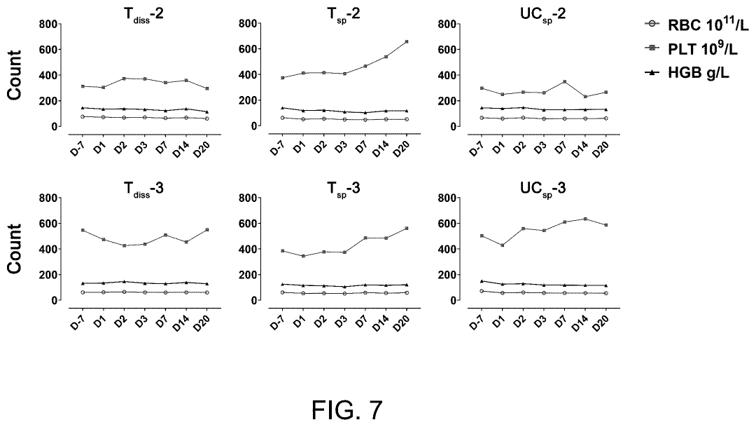

Except for the first monkey euthanized at 1-hour post-injection, the remaining two monkeys in each group showed no significant changes in blood routine results on D0 and D20. While there were individual differences in cell counts, no apparent changes were observed in the measured parameters before and after cell injection (FIG. 7).

TABLE 2
Coagulation testing of monkeys 1 week before injection

| | | | 1 W before cell injection | | | | | | | | |
|---|---|---|---|---|---|---|---|---|---|---|---|
| Cruor test | | | UC-MSCₛₚ | | | T-MSCₛₚ | | | T-MSC_{diss} | | |
| Group | Ref. | Unit | A1 | A2 | A3 | A4 | A5 | A6 | A7 | A8 | A9 |
| PT | 9-13 | Sec | 9.1 | 9.1 | 8.9 | 10.5 | 8.8 | 9.2 | 9.3 | 10 | 9.2 |
| PT INR. | 0.8-1.5 | | 0.79 | 0.79 | 0.77 | 0.91 | 0.74 | 0.8 | 0.81 | 0.87 | 0.8 |
| APTT | 20-45 | Sec | 24.1 | 24.8 | 23.4 | 23.3 | 25.1 | 25.9 | 27.5 | 27.1 | 22.5 |
| FIB | 2-4 | g/L | 1.46 | 1.32 | 2.16 | 1.93 | 2.08 | 1.96 | 1.7 | 2.06 | 2.02 |

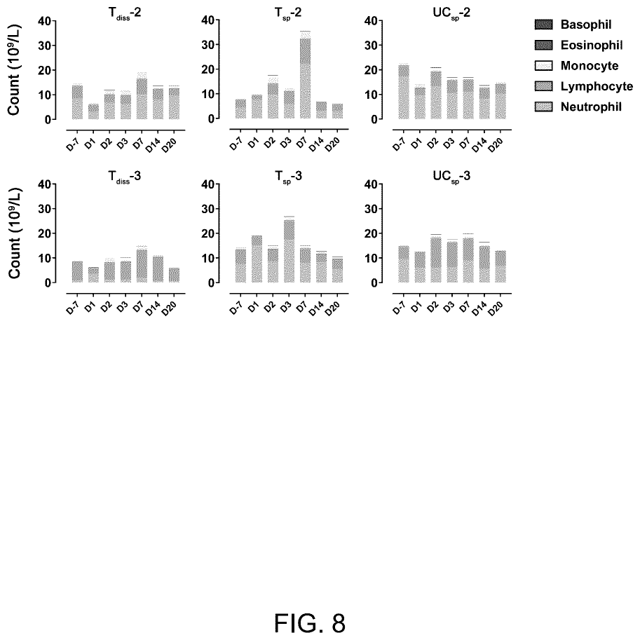

This study also included the analysis of leukocyte counts in the blood samples, including eosinophils, basophils, neutrophils, monocytes, and lymphocytes. In the T-MSCₛₚ group, the neutrophil count of two monkeys increased from D0 to D7 but subsequently returned to baseline levels. In the T-MSC_{diss} group, one monkey had a consistently lower proportion of neutrophils compared to the other monkeys. These changes in lymphocyte counts or proportions indicate that despite the immunosuppressive effect of dexamethasone, some monkeys may have exhibited more pronounced immune reactions to the injected human cells (FIG. 8).

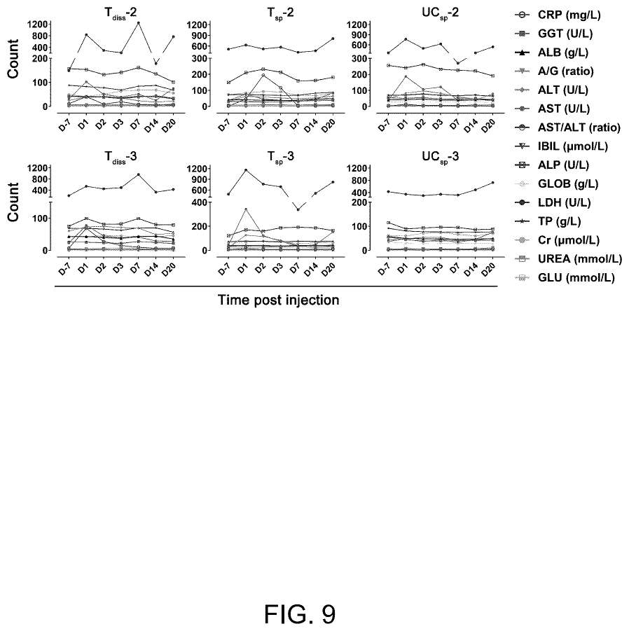

The results of blood biochemical analysis showed that most parameters remained stable, except for fluctuations in markers of tissue damage and inflammation such as lactate dehydrogenase (LDH), aspartate transaminase (AST), and C-reactive protein (CRP). As these observed fluctuations occurred in monkeys from different groups, it suggests that the transient inflammation may be associated with human cell transplantation (FIG. 9).

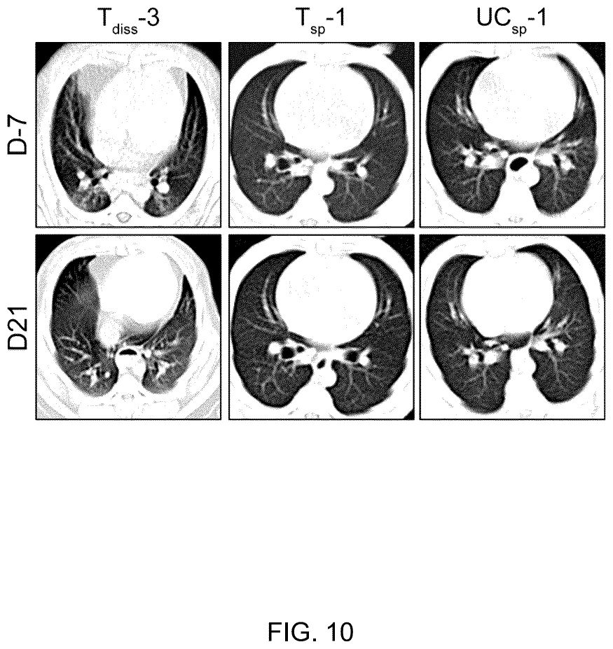

In addition, lung CT scans were performed on animals at D0 and D21 to determine if there was any occurrence of pulmonary inflammation after intravenous injection of human cells. The results showed clear lung contours, no acute exudative lesions in the lung parenchyma, and unobstructed bronchi in all animals (FIG. 10). No abnormalities were detected in the scans of the head, neck, and abdomen region as well.

### 5. Distribution of Human Cells After Injection

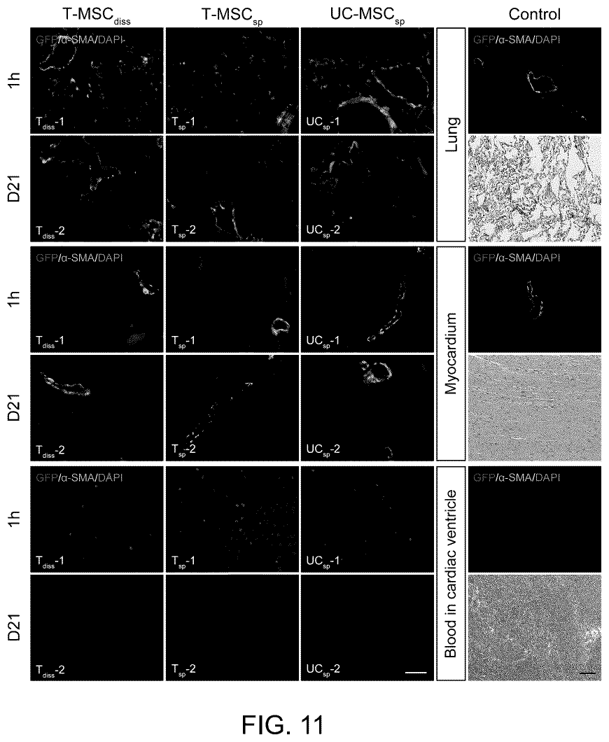

After euthanasia of the monkeys on D21, their major internal organs including the heart, liver, spleen, lungs, and kidneys were collected for histological analysis as well as nucleic acid and protein extraction. Using GFP immunofluorescence staining, it was found that one hour after injection, GFP-positive human MSCs were mainly detected in the lungs and heart. By labeling vascular smooth muscle with α-SMA, it was observed that intravenously injected MSCs could penetrate blood vessels and enter tissues, rather than just remaining within the vessels. GFP-positive cells were also found in the blood within the cardiac chambers (FIG. 11).

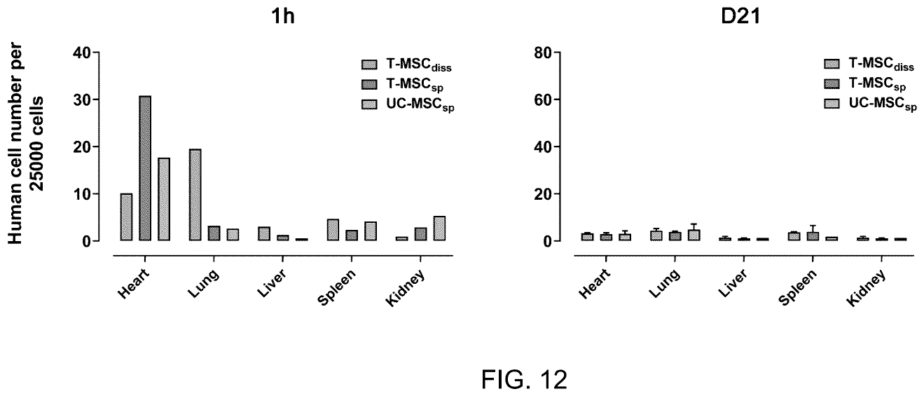

Furthermore, qRT-PCR amplification of human DNA-specific sequences in different tissues revealed that MSCₛₚ had less retention in the lungs compared to MSC_{diss} one hour after injection (FIG. 12).

6. Expression of Integrin on MSCₛₚ and MSC_{diss}

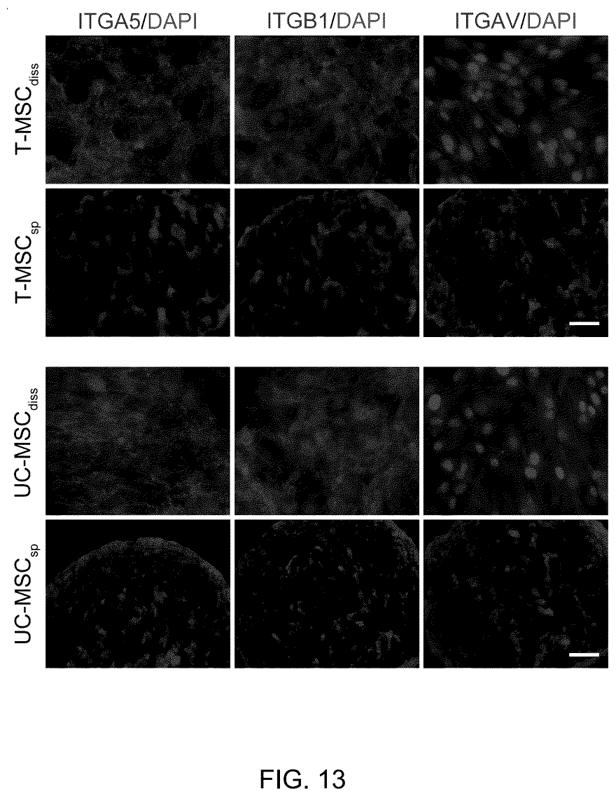

Human placental MSCs, after being cultured as a monolayer, form aggregates and express higher levels of integrin proteins on their cell surface. Therefore, they are more likely to bind to the integrin ligands expressed on the endothelial cells in the lungs after intravenous injection, resulting in enhanced lung retention. Monolayer-cultured T-MSCs also exhibit high expression of integrin proteins ITGA1, ITGB1, and ITGαv, while only some cells in T-MSCsp express these proteins (FIG. 13). This phenomenon confirms the above results: compared to monkeys receiving MSCₛₚ injections, monkeys receiving MSC_{diss} injections showed higher retention of human cells in the lungs and lower presence in the cardiac blood (FIGS. 11-12).

### 7. In Vitro Fluidic Shear Stress Test of MSC

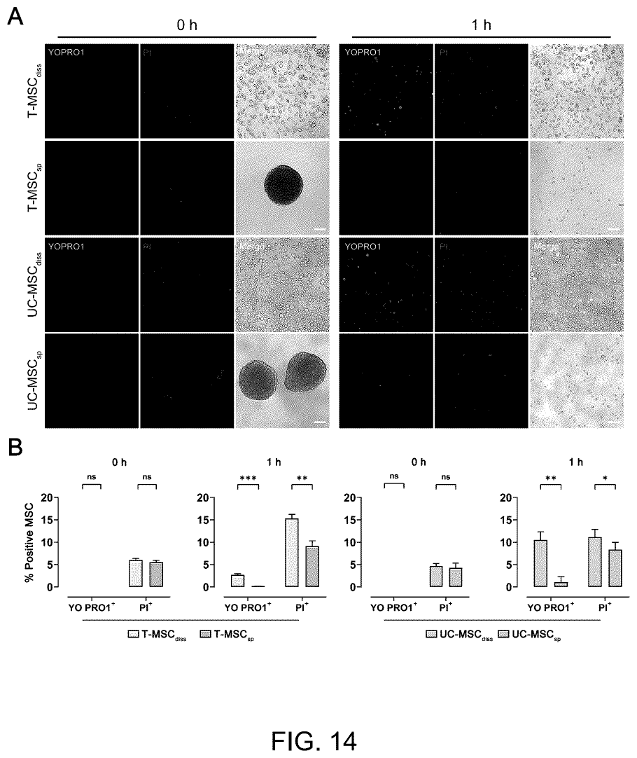

After injection, cells not only face immune system reactions from the recipient but also encounter fluid shear stress in the vasculature, which are important factors affecting the viability and therapeutic efficacy of the infused cells. In this study, UC-MSCₛₚ (approximately 300 μm) and UC-MSC_{diss} suspended in 10% FBS culture medium were injected into a microfluidic system, and the system was circulated at a speed of 60 RPM to simulate the physiological conditions of human resting heart rate. This aimed to mimic the effects of fluidic shear stress on the injected cells within the body. It was found that after 1 hour of circulation, apoptosis (YOPRO1⁺) and necrosis (PI⁺) of MSC_{diss} cells significantly increased. On the other hand, MSCₛₚ dissociated into cell clusters and individual cells after 1 hour of circulation. Although there were some PI⁺ cell fragments after dissociation, most of the cells in the cell clusters and the dissociated individual cells showed negative staining for both YOPRO1 and PI (FIG. 14).

8. Fluorescent Methacrylate Hydrogel icrospheroid for Safety Validation of Intravenous Injection

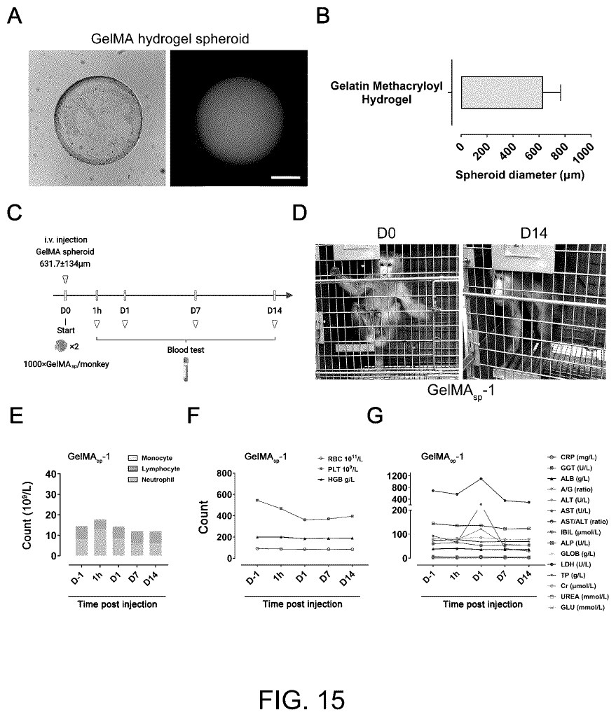

Based on the safety testing results of intravenous injection of the above-mentioned cell spheroids, it can be concluded that monkeys did not exhibit any adverse reactions and the cell spheroids demonstrated superior properties compared to traditional single cells. To further validate and expand the safety and application of spherical materials for intravenous injection, fluorescent methacrylate hydrogel microspheroid with an average size of approximately 660 μm were injected into two monkeys. Similar to the results of MSC spheroid injection (refer to FIG. 15), after 14 days of injection, the monkeys did not show any adverse reactions or physiological changes.

Biomaterial spheroids can carry drugs, bioactive molecules, or detection molecules, which can be used in animal disease models or for diagnosis and treatment of patients. They are more effective and long-lasting than individual molecules and can also carry multiple molecules to produce synergistic effects. These molecules can adhere to the surface of the spheroid, encapsulated in the core of the spheroids, or be connected to individual biomaterial molecules. Therefore, this invention provides a new approach and research method for the clinical application of cells and biomaterials.

This embodiment demonstrates that intravenous injection of MSC spheroid with a size of approximately 450 μm into non-human primate animal models is safe. Over a period of 21 days after injection, all monkeys showed no significant adverse reactions or physiological changes. Compared to monolayer-cultured MSCs, MSC spheroid have lower expressions of integrin proteins, resulting in less retention in the lungs and more presence in the bloodstream after intravenous infusion.

In addition, MSC spheroid are more resistant to fluidic shear stress compared to dissociated MSCs, thereby maintaining higher cellular viability.

In sum, the intravenous injection of cell spheroid may serve as a novel administrative approach for MSCs, reducing their retention in the lungs and prolonging their residence time and viability in the bloodstream and peripheral tissues, thereby enhancing therapeutic efficacy.

The above description is only preferred embodiments of the present invention and should not be used to limit the scope of the invention. Those skilled in the field will appreciate that various modifications and changes can be made to the present invention. Any modifications, equivalent substitutions, improvements, etc., made within the spirit and principles of the present invention should be included within the scope of the present invention's protection.

1. A method of preparing an intravenous injection agent, comprising:
providing a spherical substance selected from at least one: mesenchymal stromal cells (MSCs) spheroids and spherical materials formed by biomaterials that have a similar or comparable size to the MSC spheroids.

2. The method of claim 1, wherein the diameter of the MSC spheroids is 200 to 800 μm.

3. (canceled)

4. The method of claim 1, wherein the MSC spheroids are selected from any one of: spheroids formed by MSCs differentiated from pluripotent stem cells or spheroids formed by MSCs derived from somatic tissues.

5. The method of claim 1, wherein the biomaterials comprise at least one of: collagen, hyaluronic acid, and chondroitin sulfate.

6. The method of claim 1, wherein the subject for administrating the intravenous injection agent is a human or a non-human animal.

7. The method of claim 1, wherein the agent is used for treating autoimmune disease or inflammatory damage to tissues or organs.

8. The method of claim 7, wherein the autoimmune disease comprises multiple sclerosis.

9. The method of claim 7, wherein the inflammatory damage to tissue or organs comprises COVID-19 pneumonia.

10. The method of claim 1, wherein the intravenous injection agent further comprises an additive.

11. The method of claim 4, wherein the MSCs differentiated from pluripotent stem cells comprise those differentiated via trophoblast (T-MSCs).

12. The method of claim 4, wherein the somatic tissues are selected from at least one of: umbilical cord, adipose, or bone marrow.

13. The method of claim 6, wherein the non-human animal is selected from any one of: sheep, horse, cow, rabbit, monkey, or camel.

14. The method of claim 10, wherein the additive is selected from at least one of: physiological saline and glucose solution.
